(19) United States
(12) Patent Application Publication
(10) Pub. No.: US 2008/0306502 A1
(43) Pub. Date: Dec. 11, 2008

(54) Drive module for a device for the local piercing of a human or animal skin and a handheld

(76) Inventors: Walter Lisec, Berlin (DE); Frank Adler, Berlin (DE)

Correspondence Address: SMITH PATENT OFFICE, 1901 PENNSYLVANIA AVENUE N W, SUITE 901, WASHINGTON, DC, 20006, US

(21) Appl. No.: 12/068,957

(22) Filed: Feb. 13, 2008

(30) Foreign Application Priority Data
Feb. 14, 2007 (EP) 07003119.0
Jul. 6, 2007 (EP) 07013306.1

(51) Int. Cl. A61B 17/34

(52) U.S. Cl. 606/186

(57) ABSTRACT

The invention relates to a drive module for a device for the local piercing of a human or an animal skin, with a drive apparatus which is configured for the purpose of producing a drive rotary movement and a conversion mechanism coupled to the drive apparatus which is configured for the purpose of converting the drive movement into a coupling-connectable forward/backward movement for a needle apparatus piercing the skin locally, and which comprises a free-running functional member, wherein the functional member is coupled to a magnetic anti-turn locking element allowing a wobble or tilting movement of the functional member where, with the said anti-turn locking element, a magnetic retaining force is provided which acts against a turning of the functional member. Furthermore, the invention relates to a handheld with a drive module and a processing module in which a needle apparatus having a needle is formed and which is configured for the purpose of converting the forward/backward movement provided by the drive module into a piercing movement.

The invention relates to a drive module for a device for the local piercing of a human or an animal skin as well as a handheld.

### BACKGROUND OF THE INVENTION

Devices for the local piercing of a human or an animal skin are normally executed as handhelds. Operating personnel can use such handhelds for applying a color for a tattoo and/or permanent makeup in the zone of the skin surface. But the injection of cosmetic or medical active ingredients by way of the skin is also possible with such equipment. In addition, such equipment can be used without any substance being injected at all.

A handheld for the local piercing of a skin is known, for example, from the printed matter DE 299 19 199.0. The known handheld comprises a grip element, a needle drive, a needle and a needle nozzle, wherein at least two modules are envisaged which are detachably joined to one another and the one of the two modules is formed as a re-usable basic module with integrated needle drive. The other of the two modules is a sterilized disposable module, into which all components of the handheld are integrated that can be infected by the body fluids of a customer. In this way the handheld is provided in the form of two modules, of which one of them, namely the disposable module, can be replaced after use while the other module which comprises the needle drive can be used again. With the help of the disposable module the hygienic conditions for the application of a tattoo and/or permanent makeup are improved because all such parts are replaced which could be potentially infected by the customer's body fluid emerging during the treatment. With this method, the replacement of the entire handheld is avoided.

From the document EP 1 495 782 a drive module for a device for the local piercing of a human or an animal skin is known where a drive apparatus, with which a drive movement is producible, and a conversion mechanism coupled to the drive apparatus are envisaged, with which the drive rotary movement is converted into a coupling-connectable forward/backward movement for a needle apparatus piercing the skin locally, and a repetitive movement of the needle apparatus is enabled in this way. The conversion mechanism comprises a functional member which performs a wobble or tilting movement during the movement conversion, wherein a drive force is provided for moving a needle apparatus piercing the skin locally in a forward and backward movement. In one embodiment, the functional member is mounted free-running by means of a ball bearing. An unintentional turning of the functional member, which can be caused by the drive rotary movement, is prevented in the known device in such a way that the functional member mounted by means of a ball bearing or a projection formed thereon engage in a recess. Experience has shown during the operation of the device that this type of locking of the functional member leads to a non-inconsiderable and undesirable noise effect that is particularly caused by the changing movements of the projection within the recess.

### SUMMARY OF THE INVENTION

The object of the invention is to state and present a drive module for a device for the local piercing of a human or an animal skin as well as a handheld where the undesirable noise effect is minimized for the user during operation. Furthermore, vibrations during the operation of the device are to be reduced.

This object is solved according to the invention by a drive module according to the independent claim 1 and a handheld according to the independent claim 12.

The invention comprises the conceptual idea, involving a drive module for a device for the local piercing of a human or an animal skin, of envisaging a drive apparatus which is configured to produce a drive rotary movement and a conversion mechanism coupled to the drive apparatus where said conversion mechanism is configured for the purpose of converting the drive movement into a coupling-connectable forward/backward movement for the needle apparatus piercing the skin locally, wherein the drive mechanism comprises a free-running functional member coupled to a magnetic anti-turn locking element allowing a wobble or tilting movement of the functional member where, with the said anti-turn locking element, a magnetic retaining force is provided which acts against a turning of the functional member. With the help of the magnetic anti-turn locking element, an option for individual adaptation to various embodiments of the drive module is created for providing a sufficient retaining force which reliably prevents an unintentional turning of the functional member. With the wobble or tilting movement of the functional member, the drive rotary movement of a drive shaft at the drive apparatus is converted into a linear and repetitive movement. In order to obtain a best possible reproducibility of the forward/backward movement, a turning of the functional member should be practically or completely excluded. With the help of the magnetic anti-turn locking element, the mechanical anti-turn locking element which is known as such and which leads to considerable and undesirable noise levels in known models is supplemented or even completely replaced.

By means of a suitable selection of the magnetic retaining force the anti-turn locking element can be executed contact-free or, with the inclusion of a contact formation, between elements of the anti-turn locking element. With sufficient strength of the magnetic drawback force, the elements of the anti-turn locking element can also be kept in a spacing arrangement during operation.

A preferred further development of the invention envisages that the magnetic anti-turn locking element is formed as a non-contact anti-turn locking element which is configured to couple the magnetic retaining force onto the functional member. By means of a non-contact coupling, any noises possibly occurring with the magnetic anti-turn locking element are essentially excluded completely.

It can be envisaged with a purposeful embodiment of the invention that the magnetic anti-turn locking element is formed with one or several magnetic elements which are selectively arranged at the functional member and/or at a stationary member. If several magnet elements are envisaged, these can be arranged at the functional member as well as at the stationary member also.

The stationary member is, for example, a casing section of the drive module or a member which is solidly or detachably mounted on the casing and which can also be adjustably executed. In addition, the application of one or several magnet elements at the drive apparatus can be envisaged where the drive apparatus is preferably executed as an electric motor.

An advantageous embodiment of the invention envisages that the one or the several magnet elements are selected according to at least one structural type and are formed from the following group of structural types: permanent magnet element, electromagnet element, switch-off magnet element and, with regard to a produced magnetic force, a controllable magnet element. Permanent magnet elements have the advantage in that they provide for a magnetic field without the necessity of any further auxiliary means. An advantage of electromagnet elements lies in the fact that these can be given a task-dependent current supply. For this purpose it is necessary that a voltage is applied to the electromagnet which is executed, for example, as a coil. In the drive module the voltage required for this purpose is taken from the electric voltage supply for the drive apparatus, for example. Electromagnets are also controllable by means of a setting of the current strength.

In a preferred manner, a further development of the invention envisages that the several magnet elements comprise oppositely arranged magnet elements which are selectively arranged offset to gap. A purposeful embodiment envisages, for example, magnet elements lying opposite one another and are offset arranged to gap. However, a direct opposite arrangement can also be envisaged. In this case the arrangement of the allocated magnet elements can be in such a way that magnet elements allocated to one another have a mutual attraction and, in this way, they prevent a turning of the functional member. It can also be envisaged, either alternatively or supplementary, that magnet elements have a repelling action and a turning of the functional member is prevented as a result.

For an advantageous embodiment of the invention it can be envisaged that, with at least one magnet element formed at the functional member, at a coupling receptacle on the functional member, a magnetic coupling force is provided for the detachable coupling of a coupling member or a needle to the coupling receptacle. The at least one magnet element formed at the functional member can be arranged on the functional member in such a way that, on the rear side, a magnetic retaining force is developed in order to counteract the turning of the functional member and, in the direction of the front side of the functional member, a further magnetic force is provided by way of which the coupling member or a needle can be coupled to a coupling receptacle on the functional member. The magnetic force provided for the coupling action can act supplementary to a mechanical coupling of the needle or of the coupling member in the coupling receptacle. However, a stand-alone coupling action can also be envisaged with the help of the magnetic coupling force. For example, a head formed at the coupling member or on the needle can engage in a coupling receptacle executed as a recess and can be kept herein by means of the magnetic coupling force. The magnetic coupling force can be designed in this connection and in such a way that the coupling member or the needle can also swivel following engagement.

A further development of the invention can envisage that the functional member is located in a free-running mounting by means of a ball bearing.

With an advantageous embodiment of the invention a mechanical anti-turn locking element can be envisaged for the functional member. The magnetic and the mechanical anti-turn locks supplement each other in this embodiment. It can be envisaged in one embodiment that the mechanical anti-turn lock only unfolds an effect if the magnetic force counteracting the turning of the functional member has been overcome beforehand, which therefore would lead to a turning of the functional member without the additional locking with the mechanical anti-turn locking element. The mechanical anti-turn locking element then serves as a type of stop following the “break-through” of the magnetic force. Such a situation can occur particularly at the beginning of the operation of the device or in the case of a disturbance. The magnetic force counteracting the turning of the functional member is selected in a purposeful embodiment in such a way that, in normal operation, it otherwise remains with a non-contact or contact-free anti-turn lock which is formed by means of the magnetic anti-turn lock. For this purpose the applied magnet elements, which are available in various sizes, can be designed accordingly.

Preferably, a further development of the invention envisages that the magnetic anti-turn lock is formed as a movement with a damping effect leading to the engagement of the anti-turn locking element, through which the noise avoidance in particular is further supported. For the setting of the damping effect, the applied magnet elements can be selected according to the magnetic force available to them.

A further development of the invention can envisage that the mechanical anti-turn locking element has rear-side projections on the functional member as well as allocated counter-projections which are in engagement.

An advantageous embodiment of the invention envisages that the coupling member is arranged at a gradient angle of approx. 7° to approx. 8° to a drive shaft, on which the functional member is mounted in a free-running manner.

As follows, advantageous embodiments of the handheld for the local piercing of a human or an animal skin are described in greater detail.

It can be envisaged for a purposeful embodiment of the invention that the needle apparatus is magnetically coupled by way of a coupling mechanism to a coupling receptacle on the functional member. In this case a magnetic coupling can be envisaged between the needle apparatus, particularly also directly with the needle, and the coupling mechanism and/or between the coupling mechanism and the coupling receptacle on the functional member. A connecting rod, for example, serves as a coupling mechanism which can be provided on the end side with one or with two spherical heads.

An advantageous embodiment of the invention envisages that a coupling member encompassed by the coupling mechanism is formed, where said coupling member is selectively coupled directly to the coupling receptacle, laterally offsetting a coupling point for the take-over of the available forward/backward movement, selectively towards a middle axis. The coupling member is preferably executed as a connecting rod where, in a purposeful embodiment, spherical heads are formed on the end side which engage in allocated receptacles during the coupling action.

A further development of the invention preferably envisages that the drive module and the processing module are detachably connected.

For an advantageous embodiment of the invention, it can be envisaged that the processing module is a disposable module.

A further development of the invention can envisage that the processing module, selectively by means of the addition of one or several supplementary members, is a module type selected from the following group of module types and is correspondingly configured: tattoo module, permanent make-up module and active ingredient discharging module.

### DESCRIPTION OF PREFERRED EMBODIMENTS OF THE INVENTION

The invention is described as follows in greater detail on the basis of preferred embodiments with reference to the Figures of a drawing. These Figures show the following:

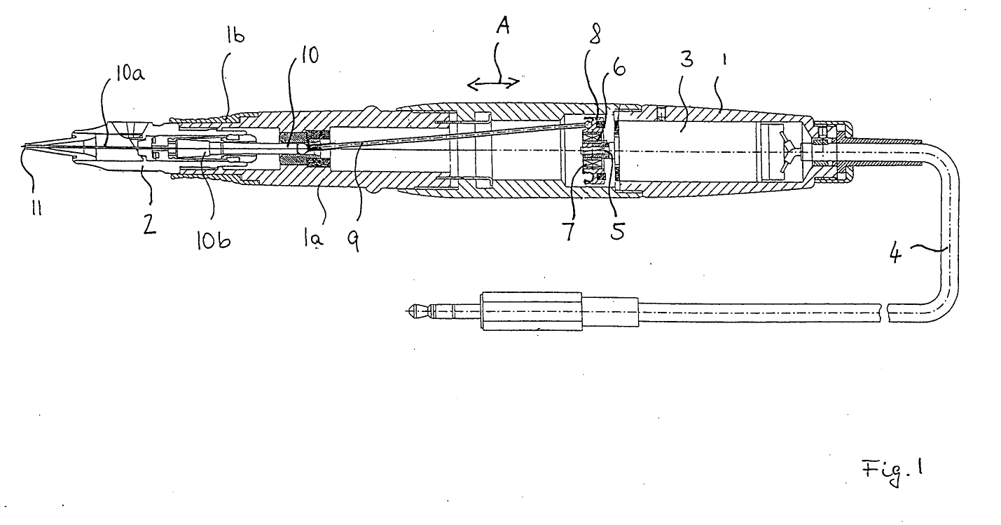

FIG. 1 is an illustration of an embodiment of a device for the local piercing of a human or an animal skin,

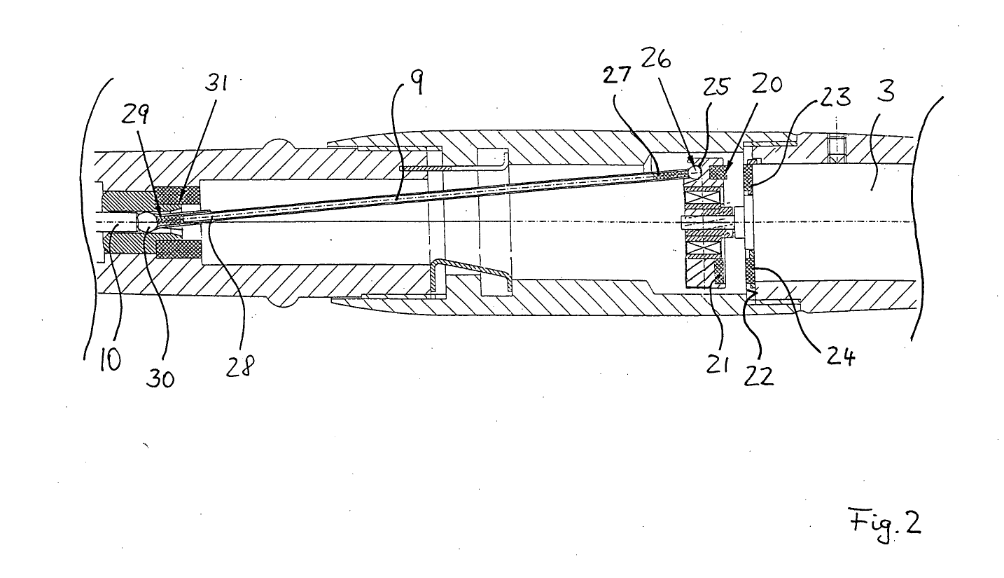

FIG. 2 is an enlarged illustration of a section of the device from FIG. 1 and

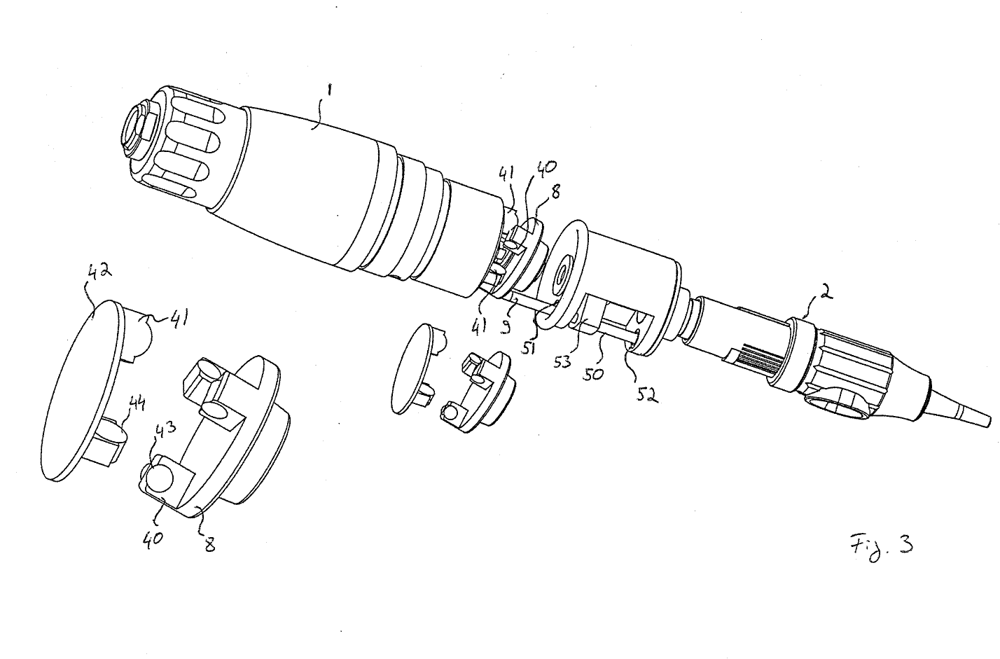

FIG. 3 is a perspective illustration of a device for the local piercing of a human or an animal skin in a further embodiment.

FIG. 1 shows an illustration of a device for the local piercing of a human or an animal skin with a drive module 1 and a processing module 2 which is detachably secured thereto. The detachable connection is preferably formed by means of a screw or an insert connection. The drive module 1 has an upper casing section 1a and, to this, an adjoining grip element 1b which can be formed in another embodiment (not shown) on the processing module 2. The drive module 1 has an integrated motor 3 which can be connected up to a voltage supply by way of a supply line 4, so that a drive rotary movement of a drive shaft 5 can be produced with the motor 3.

On the drive shaft 5 a wobble bush 6 is mounted in a stationary manner, onto which a ball bearing 7 with an inner ring and an outer ring is positioned. A functional member 7 is coupled in a free-running manner to the ball bearing 7 where said functional member 8 performs a wobble or tilting movement as a result of the drive rotary movement of the drive shaft 5 so that, onto the coupling member 9 executed as a connecting rod, a drive force for a forward/backward movement is initiated which is schematically illustrated in FIG. 1 by means of an arrow A. By way of the coupling member 9 the drive force is applied for the forward/backward movement onto a needle 10a accommodated in a needle apparatus 10. In this way the needle 10a which is held in a needle receptacle 10b, where this can be either a single needle or a needle system, is moved forward or backwards through a needle nozzle 11.

It can also be envisaged that a drive force is provided for the forward movement only. The retraction of the needle apparatus 10b is then performed with other means, for example by means of a spring membrane coupled to a needle apparatus 10b, made for example from an elastic material such as rubber or plastic. A retracting spring can also be envisaged, either as an alternative or in a supplementary manner.

The wobble bush 6, the ball bearing 7 as well as the functional member 8 coupled thereto, which is secured against turning with an anti-turn locking element described in greater detail with reference to FIG. 2, and the coupling member 9 are an integral part of a conversion mechanism with which the drive rotary movement of the drive shaft 5 is converted into a linear movement to be coupled into the needle apparatus 10. In this case, the conversion mechanism includes at least partial elements of the anti-turn locking element which is described below in greater detail.

FIG. 2 shows an enlarged illustration of a section of the device from FIG. 1. For the same features, the reference numbers as in FIG. 1 are adopted at least partially in FIG. 2.

The functional member 8 has magnet elements 20, 21 according to FIG. 2 which are inserted on the rear side into allocated recesses. On a surface 22 opposite the functional member 8, magnet elements 23, 24, allocated to the magnet elements 20, 21 are formed on the motor 3. With the help of the magnet elements 20, 21 on the functional member 8 as well as the allocated magnet elements 23, 24 on the motor 3, an anti-turn lock is formed for the free-running functional member 8. With regard to their arrangement and their magnetic configuration, the various magnet elements can be selected in such a way that magnet elements allocated to one another can either attract or repel. With the help of both configurations, a magnetic retaining force is provided which practically or completely prevents a turning of the functional member 8. In a purposeful embodiment, the magnet elements 20, 21 and the allocated magnet elements 23, 24 are arranged offset relative to one another in a home position of functional member 8 and motor 3. For example, along a peripheral line and in an alternating manner, one of the magnet elements 20, 21 and one of the allocated magnet elements 23, 24 can be formed which are preferably offset to gap.

On the front side on the functional member 8, a coupling receptacle 25 is formed as a recess in which a spherically shaped head 26 of the coupling member 9, preferably executed as a connecting rod, is mounted, wherein said coupling member 9 particularly serves the input-coupling of the drive force onto the needle apparatus 10. Adjacent to the head 26 on the coupling member 9 a further magnet element 27 is arranged which, together with the rear-side arranged magnet element 20 on the functional member 8, provides for a magnetic holding force with which the head 26 is held in the coupling receptacle 25.

According to FIG. 2 the coupling member 9 has on an end section 29 facing the needle apparatus 10 another magnet element 29 which is subsequently mounted in the end section 28 of a ball 30. The other magnet element 29 acts together with an additional magnet element 31 which encompasses fully or partially the end section 28 of the coupling member 9, at least in partial areas, and can be executed single part or multiple part. A magnetic additional force is provided in this way which counteracts an outward movement of the end section 28 from the zone of the additional magnet element 31 and, in this way, provides for a restoring force in particular. It can be envisaged, either as an alternative or as a supplement, to form the ball 30 from magnetic material or to integrate a magnet element into the ball 30.

Insofar as a material is used for the various structural elements or partial sections thereof, where said material enters into interaction magnetically with one of the magnet elements as described because of its natural properties, the individual magnet force coupling can also be formed on the basis of this interaction.

FIG. 3 shows a perspective illustration of a device for the local piercing of a human or an animal skin in a further embodiment. In FIG. 3 the same reference numbers as in FIGS. 1 and 2 are used for the same features.

The processing module 2 is detachably secured to the drive module 1. The detachable connection is preferably formed by means of a screw or an insert connection.

Unlike the embodiment in the FIGS. 1 and 2, the functional member 8 with the embodiment according to FIG. 3 is at first secured by means of a mechanical anti-turn locking element against turning during the performance of the wobble or tilting movement. For this purpose and on the functional member 8, rear-side projections 40 are formed which are arranged peripherally with equal spacing in the illustrated embodiment. The rear-side projections 40 intermesh with allocated counter-projections 41, which for their part are formed on a disk 42, and are also peripheral with equal spacing. By means of a fixation of the disk 42, through which the counter-projections 41 are stationary, the free-running functional member 8 on the drive shaft 5 is also mechanically secured against turning during operation. In the assembled condition, a spacing is set between the rear side of the functional member 8 and the front side of the disk 42 in such a way that the functional member 8 can perform the wobble or tilting movement.

As an alternative or supplement to the mechanical anti-turn locking element of the functional member 8 as already described above where the anti-turn locking element is formed directly on the functional member 8, an anti-turn locking element in mechanical formation can be formed in another manner, namely not directly on the functional member 8. It can be therefore envisaged in one embodiment (not shown) that an anti-turn locking can be achieved with a holding member (compare reference number 53 below). In this case, the contact pressure of an interacting spring is suitably selected and/or a depression for the end of the coupling member 9 on the functional member 8 is envisaged. The mechanical anti-turn locking element can, for example, be formed in the processing module 2. In one embodiment this is realized in such a way that a connecting rod, breakthroughs and a holding member (compare reference numbers 50, 51, 52 and 53 below) as well as the coupling member 9 and corresponding casing parts are moved into the processing module 2.

In addition to the mechanical anti-turn locking element, and with the embodiment according to FIG. 3, a magnetic coupling is formed with the help of a first magnet element 43 on the functional member 8 and, allocated to these, a second magnet element 44 on the disk 42 between the functional member 8 and the disk 42 which also implements the functionality of an anti-turn locking element. With a suitable material selection, the magnetic coupling can also be achieved where magnet elements are formed either only on the functional member 8 or only on the disk 42, and such magnet elements magnetically interact with allocated material zones at the other member in each individual case.

Mechanical and magnetic anti-turn locking act both against a turning of the functional member 8, wherein the magnetic coupling between the functional member 8 and the disk 42 acts with a damping effect when the rear-side projections 40 and the counter-projections 41 have a lateral surface-to-surface contact during operation. When starting up the device for the local piercing of a human or an animal skin, the repelling magnetic force between the magnet elements 43, 44 at first acts as a retaining force against the turning of the functional member 8 before the contact is established, as required, between the rear-side projections 40 and the counter-projections 41.

According to FIG. 3, a rear-side end of a connecting rod 50 lies on the front side of the functional member 8, where said rod is slidably mounted in two breakthroughs 51, 52 in the longitudinal direction of the device for local piercing. On the connecting rod 50 a holding member 53 is solidly seated which can be pressed against an initial tension, established by means of a spring, in the direction of the processing module 2, where the functional member 8 performs its wobble or tilting movement. During the course of this wobble and tilting movement of the functional member 8, the holding member 53 is then pressed also together with the connecting rod 50, because of the spring (not shown), back again into the position as illustrated in FIG. 3. With the help of this forward and backward movement of the connecting rod 50 and the holding member 53, a projecting and retracting movement of the needle tip for the repetitive piercing of the skin is obtained. The connecting rod 50 is arranged in the two breakthroughs 51, 52 in bearings inserted for this purpose which support a smooth sliding of the connecting rod 50 during the forward and backward movement. The rear-side end of the connecting rod 50 is mounted on the essentially flat-shaped surface on the front side of the functional member 8.

Independent of the various embodiments, it is envisaged that the coupling member 9 is arranged with a gradient angle of approx. 7° to approx. 8° to the drive shaft, on which the functional member 8 is mounted in a free-running manner.

The features of the invention as disclosed in this description, in the claims and in the drawing can be of significance both individually as well as in random combination for the realization of the invention in its various embodiments.

This application is based on European Patent Application Nos. EP 07003119.0 filed on Feb. 14, 2007 and EP 07013306.1 filed on Jul. 6, 2007, and the contents of which are incorporated hereinto by reference.

What is claimed is:

1. A drive module for a device for the local piercing of a human or an animal skin, with a drive apparatus which is configured for the purpose of producing a drive rotary movement and a conversion mechanism coupled to the drive apparatus which is configured for the purpose of converting the drive movement into a coupling-connectable forward/backward movement for a needle apparatus piercing the skin locally, and which comprises a free-running functional member, wherein the functional member is coupled to a magnetic anti-turn locking element allowing a wobble or tilting movement of the functional member where, with the said anti-turn locking element, a magnetic retaining force is provided which acts against a turning of the functional member.

2. The drive module according to claim 1, wherein the magnetic anti-turn locking element is formed as a non-contact anti-turn locking element which is configured for the purpose of coupling the magnetic retaining force in a non-contacting manner onto the functional member.

3. The drive module according to claim 1, wherein the magnetic anti-turn locking element is formed with one or several magnet elements which are selectively arranged on the functional member and/or on a stationary member.

4. The drive module according to claim 3, wherein the one or the several magnet elements are selected according to at least one structural type and are formed from the following group of structural types: permanent magnet element, electromagnet element, switch-off magnet element and, with regard to a produced magnetic force, a controllable magnet element.

5. The drive module according to claim 3, wherein the several magnet elements comprise oppositely arranged magnet elements which are selectively arranged offset to gap.

6. The drive module according to claim 3, wherein, with at least one magnet element formed on the functional member on a coupling receptacle on the functional member, a magnetic coupling force is provided for the detachable coupling of a coupling member or a needle apparatus to the coupling receptacle.

7. The drive module according to claim 1, wherein the functional member is mounted in a free-running manner by means of a ball bearing.

8. The drive module according to claim 1, wherein a mechanical anti-turn locking element for the functional member.

9. The drive module according to claim 8, wherein the magnetic anti-turn locking element is formed with a damping effect for the movement for the intermesh of the mechanical anti-turn locking element.

10. The drive module according to claim 8, wherein the mechanical anti-turn locking element has rear-side projections on the functional member as well as allocated counter-projections which are intermeshing.

11. The drive module according to claim 6, wherein the coupling member is arranged with a gradient angle of approx. 7° to approx. 8° to a drive shaft, on which the functional member is mounted in a free-running manner.

12. A handheld for the local piercing of a human or an animal skin, with a drive module which comprises a free-running functional member, wherein the functional member is coupled to a magnetic anti-turn locking element allowing a wobble or tilting movement of the functional member where, with the said anti-turn locking element, a magnetic retaining force is provided which acts against a turning of the functional member, and a processing module in which a needle apparatus having a needle is formed and which is configured for the purpose of converting the forward/backward movement provided by the drive module into a piercing movement.

13. The handheld according to claim 12, wherein the needle apparatus is magnetically coupled directly to a coupling receptacle on the functional member.

14. The handheld according to claim 12, wherein the needle apparatus is magnetically coupled to a coupling receptacle on the functional member by way of a coupling mechanism.

15. The handheld according to claim 14, wherein a coupling member encompassed by the coupling mechanism is formed, where said coupling member is selectively coupled directly to the coupling receptacle, laterally offsetting a coupling point for the take-over of the available forward/backward movement, selectively towards a middle axis.

16. The handheld according to claim 12, wherein the drive module and the processing module are detachably connected.

17. The handheld according to claim 16, wherein the processing module is a disposable module.

18. The handheld according to claim 12, wherein the processing module, selectively by means of the addition of one or several supplementary members, is a module type selected from the following group of module types and is correspondingly configured: tattoo module, permanent make-up module and active ingredient discharging module.
